(19) United States
(12) Patent Application Publication
(10) Pub. No.: US 2026/0074074 A1
(43) Pub. Date: Mar. 12, 2026

(54) CROSS-DOMAIN MULTI-MODAL TIME SERIES ANNOTATION FOR MEDICAL DECISION MAKING

(71) Applicant: NEC Laboratories America, Inc., Princeton, NJ (US)

(72) Inventors: Zhengzhang Chen, Princeton Junction, NJ (US); Haifeng Chen, West Windsor, NJ (US); Junxiang Wang, Plainsboro, NJ (US); Yanchi Liu, Princeton, NJ (US); LuAn Tang, Cranbury, NJ (US); Minhua Lin, State College, PA (US)

(21) Appl. No.: 19/323,489

(22) Filed: Sep. 9, 2025

Related U.S. Application Data
(60) Provisional application No. 63/692,761, filed on Sep. 10, 2024.
(60) Provisional application No. 63/704,669, filed on Oct. 8, 2024.

(51) Int. Cl. G16H 50/20; G16H 10/60

(52) U.S. Cl. CPC G16H 50/20; G16H 10/60

(57) ABSTRACT

Methods and systems include generating general annotations for input time series data based on annotations from one or more source domains. Domain-specific annotations are generated for the input time series based on annotations from a target domain and based on the general annotations. An action is performed responsive to the domain-specific annotations and the general annotations.

### RELATED APPLICATION INFORMATION

This application claims priority to U.S. Patent Application No. 63/692,761, filed on Sep. 10, 2024, and to U.S. Patent Application No. 63/704,669, filed on Oct. 8, 2024, each incorporated herein by reference in its entirety.

### BACKGROUND

### Technical Field

The present invention relates to time series annotation and, more particularly, to cross-domain multi-modal annotation.

### Description of the Related Art

Time series data is ubiquitous across domains such as manufacturing and healthcare. High-quality annotations are needed to effectively understand time series, making them useful in downstream tasks, but obtaining such annotations is costly and time-consuming. This task is further complicated when precise and domain-specific terminology is needed. Existing approaches to the automatic generation of annotations fail to create high-quality annotations, capturing generic labels or only basic patterns. Further there is a risk of hallucination and mislabeling.

### SUMMARY

A method includes generating general annotations for input time series data based on annotations from one or more source domains. Domain-specific annotations are generated for the input time series based on annotations from a target domain and based on the general annotations. An action is performed responsive to the domain-specific annotations and the general annotations.

A system includes a hardware processor and a memory that stores a computer program. When executed by the hardware processor, the computer program causes the hardware processor to generate general annotations for input time series data based on annotations from one or more source domains, to generate domain-specific annotations for the input time series based on annotations from a target domain and based on the general annotations, and to perform an action responsive to the domain-specific annotations and the general annotations.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of cross-domain multi-modal time series annotation, in accordance with an embodiment of the present invention;

FIG. 2 is a block diagram of a general annotation agent, in accordance with an embodiment of the present invention;

FIG. 3 is a block diagram of a domain-specific annotation agent, in accordance with an embodiment of the present invention;

FIG. 4 is a block/flow diagram of a method for annotating time series data, in accordance with an embodiment of the present invention;

FIG. 5 is a block diagram of a healthcare facility that uses cross-domain multi-modal time-series annotation to assist in medical decision making, in accordance with an embodiment of the present invention;

FIG. 6 is a block diagram of a computing device for annotating time series data, in accordance with an embodiment of the present invention;

FIG. 7 is a diagram of an exemplary neural network architecture that can be used to implement part of a policy network, in accordance with an embodiment of the present invention; and

FIG. 8 is a diagram of an exemplary deep neural network architecture that can be used to implement part of a policy network, in accordance with an embodiment of the present invention.

### DETAILED DESCRIPTION OF PREFERRED EMBODIMENTS

Annotations for time series data may be automatically generated by drawing knowledge from existing annotations across multiple source domains. The knowledge may be transferred to a target domain that has only a limited number of annotations. Time series data is interpreted across various fields using either common or domain-specific language. Given abundant annotations from the source domains and few annotations from the target domain, both time-series-wise and text-wise knowledge is used to generate accurate and contextually appropriate annotations for the target domain.

The present embodiments extract common knowledge from source domains and learn domain-specific jargon from limited target-domain annotations. To this end, a multi-agent system is used for both general and domain-specific time series annotations. A general annotation agent focuses on capturing common patterns and knowledge across domains to generate annotations that can be understood by general users. To learn common knowledge from multiple domains, the general agent employs a time series feature extractor and a text-wise feature extractor to extract both time-series-wise and text-wise features from time series and domain-specific annotations from multiple source domains. To ensure important features and included in the general annotations, large language model (LLM)-based selection and reinforcement learning-based selection are used to effectively and efficiently select the top-k most important time-series-wise and text-wise features. This selection ranks the features.

The domain-specific agent leverages limited target-domain annotations to learn and generate annotations for specific domains using domain-specific terminologies. It incorporates a domain-specific term extractor to learn jargon from the limited target-domain annotations. An annotation reviewer maintains consistency between the general annotations and the domain-specific annotations.

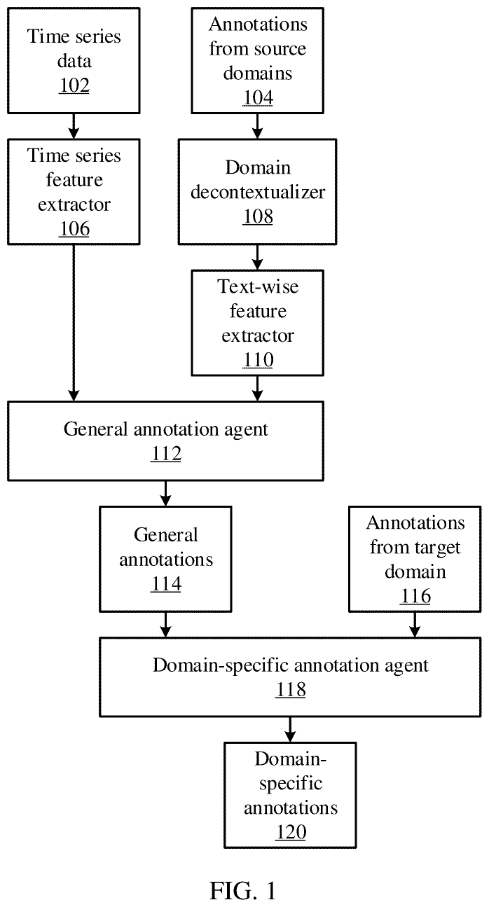

Referring now to FIG. 1, an annotation system is shown. Time series data 102 and domain-specific annotations from source domains 104 are used to generate features for a general annotation agent 112. A time series feature extractor 106 extracts time series-wise features from the time series data 102. A domain decontextualizer 108 extracts decontextualized annotations from the source domain annotations 104 by removing domain-specific language from text and a text-wise feature extractor 110 extracts text-wise features. The time series-wise features and the test-wise features are input to the general annotation agent 112, which generates general annotations 114.

The general annotations 114 and domain-specific annotations from the target domain 116 are used as inputs to a domain-specific annotation agent 118. The domain-specific annotation agent 118 then generates domain-specific annotations 120.

Given a time series

$${\{ {x}_{i} \}}_{i = 1}^{L} = ( {x}_{1} , , … , {x}_{L} ) ,$$

where L is a number of past time stamps and xᵢ=(x₁ᵢ, . . . , xₘᵢ)^{T}∈ [symbol] ^{C} represents data from C different channels at timestamp i. The time-series annotation generates a descriptive annotation or interpretation that captures significant patterns, trends, or features in the time series data 102. For simplicity a univariate time series x=(x₁, . . . , x_{L}) is used herein, where each xᵢ∈ [symbol] , ∀i∈ L. Those having ordinary skill in the art can extend this to multivariate time series data.

General annotations e_{g} are domain-agnostic, while domain-specific annotations eₛ include language specific to particular domains. The general annotations 114 highlight patterns that are significant across domains, regardless of the specific field. These annotations are useful for generic analysis and can serve as a baseline for further domain-specific investigation. The domain-specific annotations 120 leverage specialized knowledge to identify patterns that are meaningful only within the context of that domain, which may help with domain-specific downstream tasks, such as medical decision making in healthcare or fault detection in engineering systems.

The time series feature extractor 106 identifies the time series-wise features Fₜ for each channel c∈C as

$${F}_{t} = \{ {f}_{t}^{1} , … , {f}_{t}^{n} \} = {ℳ}_{r} ( x ) , where {f}_{t}^{i}$$

is the i^{th} extracted time series-wise feature. For multivariate time series data, some intervariable features, such as a Pearson correlation, may also be used.

Existing source domain annotations 104 may include time series features that can help to interpret the time series data 102. To extract this text-wise knowledge from the source domains, the domain decontextualizer 108 converts the domain-specific annotations from the multiple source domains into generalized annotations by removing domain-specific terminology, making it easier to extract the common knowledge. Given a domain-specific annotation eₛ in the domain dᵢ, an LLM may be used to write the decontextualized annotation e_{d} based on eₛ and dᵢ as e_{d}= [symbol] _{d}(p_{de}(eₛ, dᵢ), where [symbol] _{d} is the domain decontextualization LLM and p_{de} is the prompt of domain decontextualization for the LLM.

After domain decontextualization 108, an LLM [symbol] may be used as the text-wise feature extractor 110 to extract text-wise common knowledge from multiple source domains that are not explored from the time-series perspective. Formally, given a set of decontextualized annotations

$${\{ {e}_{d}^{i} \}}_{i = 1}^{{n}_{d}}$$

and a text-wise feature extractor [symbol] , the extracted text-wise features may be denoted as

$${ℳ}_{l} ( {p}_{l} ( {\{ {e}_{d}^{i} \}}_{i = 1}^{{n}_{d}} )$$

where [symbol] is the prompt of text-wise feature extraction.

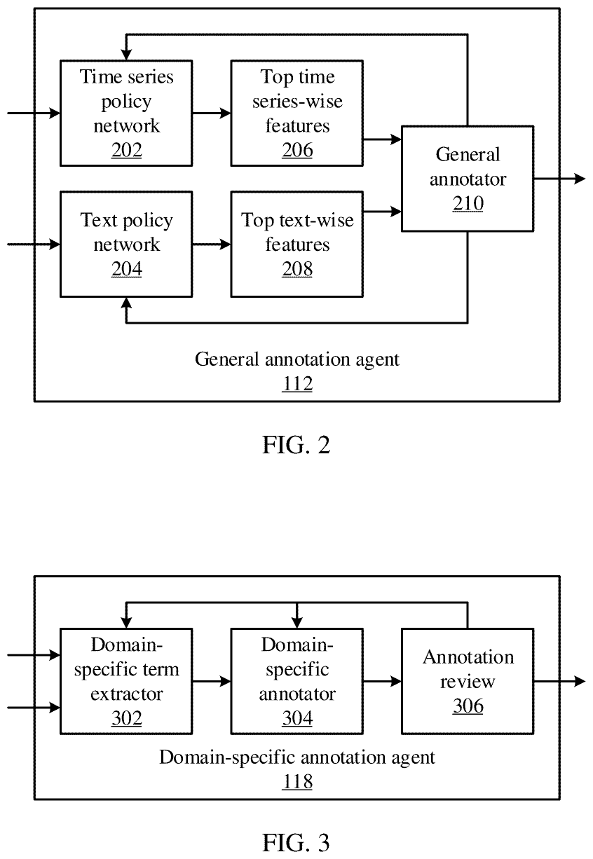

Referring now to FIG. 2, additional detail is shown on the general annotation agent. The time series-wise features from the time series feature extractor 106 are input to a time series policy network 202. The text-wise features from the text-wise feature extractor 110 are input to a text policy network 204. The respective policy networks select the top-k time series-wise features 206 and the top-k text-wise features 208, where k is a hyperparameter. A general annotator 210 produces the general annotations 114 for output based on the selected features.

Time series feature extractor 106 and text-wise feature extractor 110 may generate many features. However, the generated annotations 114 should not be overly long as it could hinder understanding by users. A word limit may therefore be set for the general annotations 114, and this word limit prevents using all of the extracted features. To ensure that the general annotations 114 highlight the most important patterns, the time series policy network 202 and the text policy network 204 select the most important features.

Reinforcement learning may be used to continuously adapt to changes to new data, for example from a new domain or additional data from a previously seen domain. The time series policy network 202 and the text policy network 204, represented as [symbol] and [symbol] respectively, select the top-k most important time series-wise features 206 and the top-k text-wise features 208. The first three layers of each policy network may be taken from a small LLM and kept fixed. For example, the first three layers of GPT-2 may be used, followed by a trainable multi-head attention layer with two heads, and then the language model head layer from GPT-2.

To train the policy networks 202 and 204, only the multi-head attention is trained, while the parameters of the other layers are kept fixed. For the time series policy network 202, given a state s, the mean of logits is determined for all

$${n}_{name}^{i} tokens \{ {y}_{1}^{i} , … , {y}_{{n}_{name}^{i}}^{i} \}$$

in the names of the time series-wise features. This is performed for all names of the time series-wise features, yielding q_{s,name}_{ᵢ}, providing a vector of action-values (Q-values) qₛ={q_{s,name}_{₁}, . . . , q_{s,name}_{ₙₜ}}. A softmax function may be used to obtain a probability distribution over the feature names.

During exploration (e.g., while training), the action at is obtained by sampling time series-wise features

$${\{ {f}_{t}^{i} \}}_{i = 1}^{k} ,$$

with the top-k highest values from this distribution. At each timestamp a new set of top-k most important time series-wise features are selected. The selected features are then sent to a selection LLM to obtain the corresponding score sⱼ, ∀j∈ {1, . . . , k}. The agent receives a reward at each timestamp t following:

$${r}_{t} = \{ \begin{matrix}{\sum }_{i = 1}^{k} {s}_{i} , {s}_{i} \geq τ \\ - 0.5 otherwise\end{matrix}$$

where τ is a scoring threshold that prevents selecting unimportant features. The text policy network 204 can be trained in a similar way. After training the policy networks 202 and 204, when new features are introduced, the policy networks can be incrementally updated instead of completely retraining them.

In some embodiments, instead of reinforcement learning policy networks, the selection of the top time series-wise features 206 and the top text-wise features 208 may be performed by an LLM. LLMs are capable of performing feature selection from textual annotations using an LLM-generated feature importance score-based selection that leverages the abilities of the LLM. If a feature is explicitly or implicitly mentioned more frequently in the annotations than other features, then it should be considered more important and should be assigned a higher importance score. An explicitly mentioned feature should be considered more important than an implicitly mentioned feature.

A pretrained LLM [symbol] _{select} for feature selection may be prompted with the domain-decontextualized annotations

$${e}_{{d}_{i} = 1}^{{i}^{{n}_{d}}}$$

and the extracted time series-wise features

$${\{ {f}_{t}^{i} \}}_{i = 1}^{{n}_{i}} ,$$

respectively, to obtain sets of numerical feature importance scores sₜ=[s₁, . . . , sₙ_{ₜ}] and sₗ=[s₁, . . . , sₙ_{ₗ}] for

$${\{ {f}_{t}^{i} \}}_{i = 1}^{{n}_{t}} and {\{ {f}_{t}^{i} \}}_{i = 1}^{{n}_{l}}$$

respectively. Formally, this is denoted as:

$${s}_{j} = {select}_{} ( {p}_{score} ( {f}_{t}^{j} , {e}_{{d}_{i} = 1}^{{i}^{{n}_{d}}} ) , ∀ j ∈ \{ 1 , … , {n}_{t} \} \\ {s}_{k} = {select}_{} ( {p}_{score} ( {f}_{t}^{k} , {e}_{{d}_{i} = 1}^{{i}^{{n}_{d}}} ) , ∀ j ∈ \{ 1 , … , {n}_{l} \}$$

where p_{score} is the prompt of scoring feature importance. Higher values for sⱼ, sₖ∈ [symbol] ⁺ indicate that the features

$${f}_{t}^{j} and {f}_{t}^{k}$$

explicitly or implicitly appear more frequently in the domain-decontextualized annotations. A higher weight is assigned to features that are explicitly mentioned when calculating sⱼ and sₖ via the LLM [symbol] _{select}.

After selecting the most important features, a general annotator 210 analyzes the selected features to generate the general annotations 114. An LLM may be used as the general annotator 210, interpreting the given time series data 102 based on the selected features. Given a time series

$$\{ {x}_{{1}_{i = 1}}^{L} \}$$

and the selected time series-wise features

$${\{ {f}_{t}^{i} \}}_{i = 1}^{{n}_{t}}$$

and selected text-wise features

$${\{ {f}_{t}^{i} \}}_{i = 1}^{{n}_{l}} ,$$

the general annotation e_{g} may be determined as:

$${e}_{gen} = {gen}_{} ( {p}_{gen} ( \{ {x}_{{1}_{i = 1}}^{L} \} , {\{ {f}_{t}^{i} \}}_{i = 1}^{{n}_{t}} , {\{ {f}_{t}^{i} \}}_{i = 1}^{{n}_{l}} )$$

where p_{gen} is the prompt instructing the LLM to generate general annotations 114. By highlighting the signal from the selected common knowledge, the general annotations 114 contain richer patterns that may be overlooked by directly applying LLMs.

Referring now to FIG. 3, additional detail on the domain-specific annotation agent 118 is shown. The domain-specific annotation agent 118 receives the general annotations 114 and the annotations from the target domain 116 as inputs and a domain-specific term extractor 302 operates to identify terminology that is specific to the target domain. Given limited domain-specific annotations

$${\{ {e}_{t}^{i} \}}_{i = 1}^{{n}_{{e}_{t}}}$$

from the target domain, an LLM [symbol] ₑₓₜ is prompted to extract a set of domain-specific terms:

$${\{ {i}^{} \}}_{i = 1}^{{n}_{}} = {ext}_{} ( {p}_{ext} ( {\{ {e}_{t}^{i} \}}_{i = 1}^{{n}_{{e}_{t}}} ) )$$

where [symbol] is the number of extracted domain-specific terms and pₑₓₜ is a prompt instructing the LLM to perform term extraction.

A domain-specific annotator 304 ensures alignment between the domain-specific annotations and the general annotations, using an LLM [symbol] ₛₚₑ to apply extracted domain-specific terms

$${\{ {i}^{} \}}_{i = 1}^{{n}_{}}$$

to the general annotations e_{g}, converting them into target-domain annotations eₜ:

$${e}_{t} = {spe}_{} ( {p}_{spe} ( {e}_{g} , {\{ {i}^{} \}}_{i = 1}^{{n}_{}} ) )$$

where pₛₚₑ is a prompt instructing the LLM to generate domain-specific annotations.

Annotation review 306 improves the jargon extraction and the alignment between domain-specific annotations and general annotations, further ensuring the quality of domain-specific annotations. The annotation review 306 may use an LLM [symbol] ᵣₑᵥ to examine the domain-specific annotations and the extracted domain-specific terms, providing feedback e_{f} to the extractor 302 and the annotator 304:

$${e}_{f} = {rev}_{} ( {p}_{rev} ( {e}_{g} , {e}_{t} , {\{ {i}^{} \}}_{i = 1}^{{n}_{}} ) )$$

where pᵣₑᵥ is a prompt that instructs the LLM to review annotations. This process ensures the extraction of more precise domain-specific terms and better alignment between general and domain-specific annotations. Upon receiving feedback from the reviewer 306, the extractor 302 refines its extraction results and the annotator 304 enhances its annotations based on the feedback and the newly extracted terms. The output is the set of domain-specific annotations 120.

For the domain-specific term extractor 302, the feedback guides refinements to term identification rules and ranking logic, enabling the extractor to improve precision and recall in identifying relevant terminology. For the domain-specific annotator, the feedback informs adjustments to annotation strategies, contextual phrasing, and consistency rules, allowing it to produce more accurate, contextually aligned, and domain-authentic annotations. Over time, this iterative process enhances both the completeness of term coverage and the quality of domain-specific language in the system's outputs.

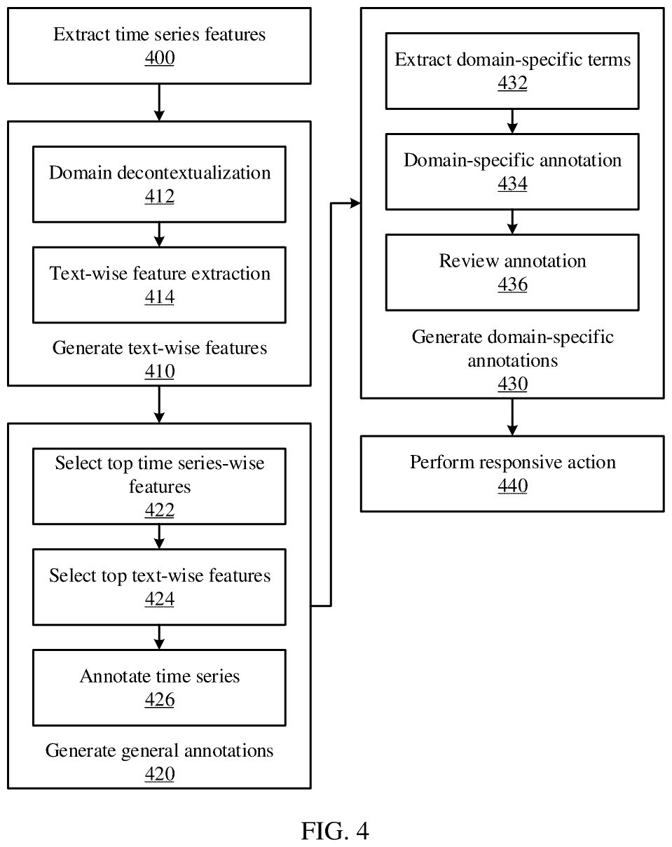

Referring now to FIG. 4, a method for performing cross-domain multi-modal time series annotation is shown. Block 400 extracts time series features from the input time series data 102 and block 410 extracts features from the source domain annotations 104. The generation of text-wise features 410 includes domain decontextualization 412, for example removing domain-specific terms, and block 414 extracts the text-wise features.

Block 420 generates the general annotations 114, using any appropriate mechanism to rank and select the top-k features from the time series features and the text-wise features. The selection 422 of the top time-series-wise features and the selection 424 of the top text-wise features may be performed using respective policy networks or may be performed using an LLM. Block 426 generates general annotations for the time series according to the selected features.

Block 430 generates domain-specific annotations, first extracting 432 domain-specific terms from the annotations 116 from the target domain and then performing domain-specific annotation 434. Block 436 reviews the annotations and generates feedback to update the extraction 432 and annotation 434.

Block 440 performs an action based on the domain-specific annotations 120 and the general annotations 114. For example, the target domain may relate to a medical domain dealing with a particular condition or medical discipline and may generate diagnoses for a patient based on time series data that represents their health condition. In such embodiments, the action may include a treatment action. For example, the annotation may label the time series data as indicating a particular diagnosis, and the action may be a treatment to address that diagnosis.

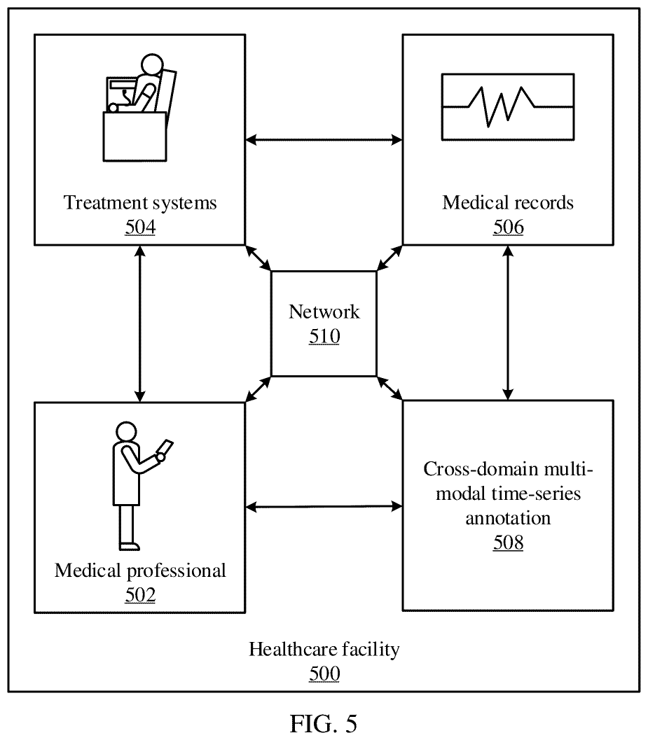

Referring now to FIG. 5, a diagram of time series analysis is shown in the context of a healthcare facility 500. Cross-domain multi-modal time series annotation 508 may be used to analyze time series data relating to a patient's condition. The time series data may be drawn from the patient's medical records 506 or directly from treatment systems 504, and may include measurements, test results, and other data that changes over time.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the cross-domain multi-modal time series annotation 508, the medical professionals 502 may then make medical decisions about patient healthcare suited to the patient's needs. For example, the medical professionals 502 may make a diagnosis of the patient's health condition and may prescribe particular medications, surgeries, and/or therapies that are appropriate to the stage of a disease.

The different elements of the healthcare facility 500 may communicate with one another via a network 510, for example using any appropriate wired or wireless communications protocol and medium. Thus the cross-domain multi-modal time series annotation 508 receives data from treatment systems 504, medical professionals 502, and from medical records 506, and labels the time series data using labels that are derived from a domain-specific dataset. Medical databases may have much sparser annotations than general purpose training databases, but accuracy in applying labels for health conditions has high stakes. The cross-domain multi-modal time series annotation 508 therefore generates labels that accurately reflect both the knowledge derived from the general purpose databases as well as the information from the more limited annotations of the medical databases. The cross-domain multi-modal time series annotation 508 may further coordinate with treatment systems 504 in some cases to automatically administer or alter a treatment. For example, if the cross-domain multi-modal time series annotation 508 generates a label for the time series data that indicates the patient may have a particular health condition, the information system may trigger a change in treatment, such as initiating or halting the administration of a medication.

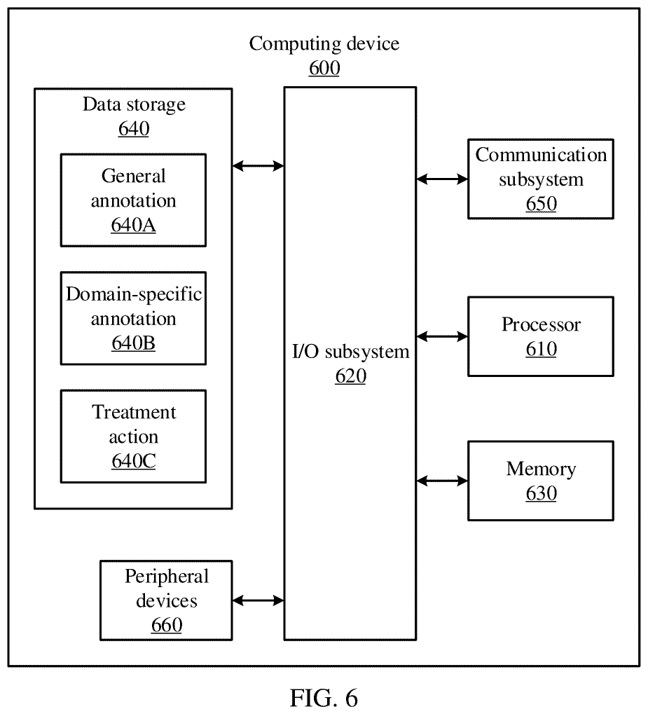

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for general annotation, 640B for domain-specific annotation, and/or 640C for performing a treatment action. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

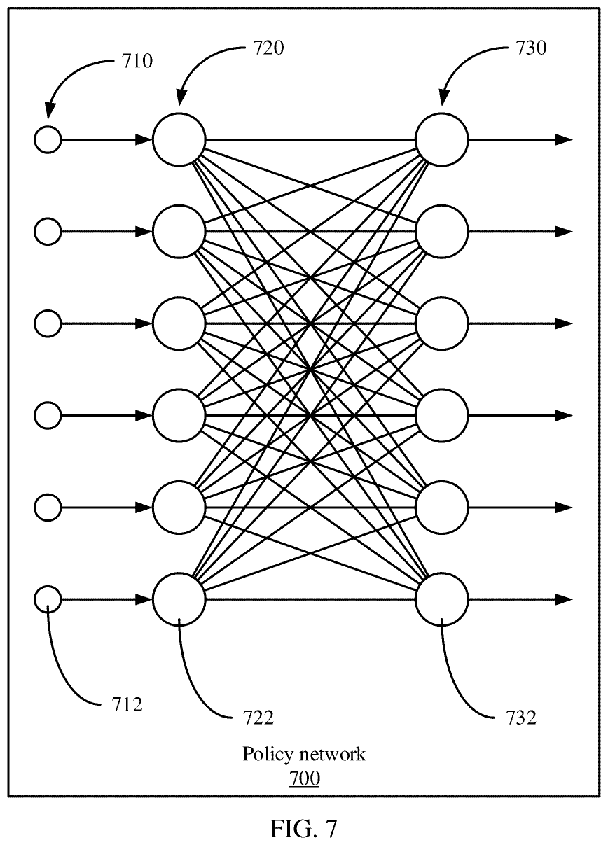

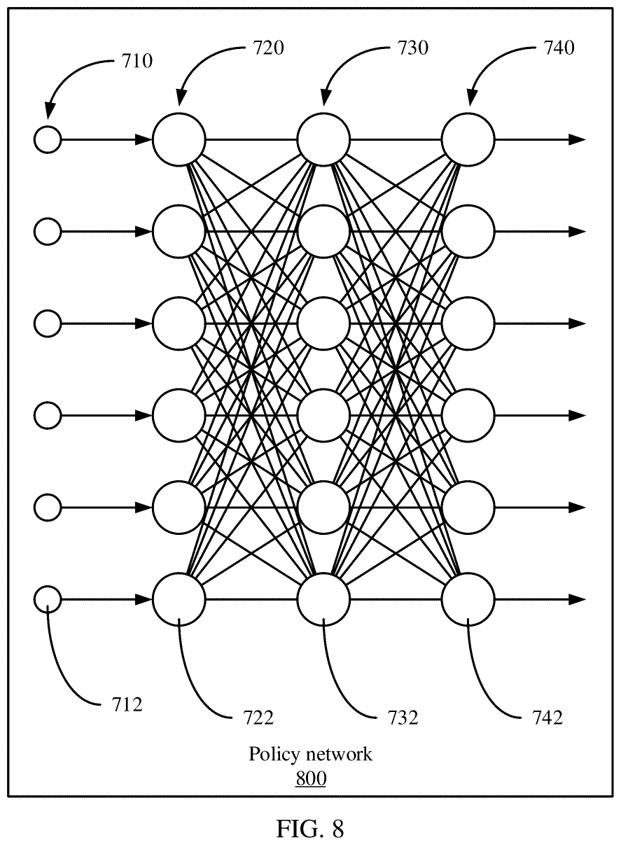

Referring now to FIGS. 7 and 8, exemplary neural network architectures are shown, which may be used to implement parts of the present machine learning models, such as the policy networks 700/800. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
generating general annotations for input time series data based on annotations from one or more source domains;
generating domain-specific annotations for the input time series based on annotations from a target domain and based on the general annotations; and
performing an action responsive to the domain-specific annotations and the general annotations.

2. The method of claim 1, further comprising extracting time series features from the input time series data and extracting text-wise features from the annotations from the source domains, wherein generating the general annotations is based on the extracted time series features and the extracted text-wise features.

3. The method of claim 2, wherein generating the general annotations includes selecting a set of highest ranked time series features from the extracted time series features and selecting a set of highest ranked text-wise features from the extracted text-wise features using respective policy networks.

4. The method of claim 3, further comprising updating the respective policy networks using the general annotations as feedback.

5. The method of claim 2, wherein generating the general annotations includes selecting a set of highest ranked time series features from the extracted time series features and selecting a set of highest ranked text-wise features from the extracted text-wise features using a machine learning model implementing a large language model.

6. The method of claim 2, wherein extracting the text-wise features includes removing domain-specific terms.

7. The method of claim 1, wherein generating the domain-specific annotations includes extracting domain-specific terms based on the annotations from the target domain, further comprising updating domain-specific term extraction based on feedback from the domain-specific annotations.

8. The method of claim 1, wherein the time series data includes medical information about a patient.

9. The method of claim 8, wherein the action includes identifying a condition of the patient indicated by the time series data to assist in medical decision making.

10. The method of claim 8, wherein the action includes automatically triggering a treatment action based on the domain-specific annotations.

11. A system, comprising:
a hardware processor; and
a memory that stores a computer program which, when executed by the hardware processor, causes the hardware processor to:
generate general annotations for input time series data based on annotations from one or more source domains;
generate domain-specific annotations for the input time series based on annotations from a target domain and based on the general annotations; and
perform an action responsive to the domain-specific annotations and the general annotations.

12. The system of claim 11, wherein the computer program further causes the hardware processor to extract time series features from the input time series data and extract text-wise features from the annotations from the source domains, wherein generation of the general annotations is based on the extracted time series features and the extracted text-wise features.

13. The system of claim 12, wherein generation of the general annotations includes selecting a set of highest ranked time series features from the extracted time series features and selecting a set of highest ranked text-wise features from the extracted text-wise features using respective policy networks.

14. The system of claim 13, wherein the computer program further causes the hardware processor to update the respective policy networks using the general annotations as feedback.

15. The system of claim 12, wherein generation of the general annotations includes selecting a set of highest ranked time series features from the extracted time series features and selecting a set of highest ranked text-wise features from the extracted text-wise features using a machine learning model implementing a large language model.

16. The system of claim 12, wherein extraction of the text-wise features includes removing domain-specific terms.

17. The system of claim 11, wherein generation of the domain-specific annotations includes extracting domain-specific terms based on the annotations from the target domain, wherein the computer program further causes the hardware processor to update domain-specific term extraction based on feedback from the domain-specific annotations.

18. The system of claim 11, wherein the time series data includes medical information about a patient.

19. The system of claim 18, wherein the action includes identifying a condition of the patient indicated by the time series data to assist in medical decision making.

20. The system of claim 18, wherein the action includes automatically triggering a treatment action based on the domain-specific annotations.
